(19) United States
(12) Patent Application Publication
(10) Pub. No.: US 2019/0142639 A1
(43) Pub. Date: May 16, 2019

(54) ATTACHMENT SYSTEM FOR A LENS TO GOGGLES

(71) Applicant: FOX HEAD, INC., Irvine, CA (US)

(72) Inventors: David L. DURHAM, San Clemente, CA (US); Dennis TAN, Long Beach, CA (US)

(73) Assignee: Fox Head, Inc., Irvine, CA (US)

(21) Appl. No.: 16/095,926

(22) PCT Filed: Mar. 22, 2017
(86) PCT No.: PCT/US2017/023668

§ 371 Date: Oct. 23, 2018

Related U.S. Application Data
(60) Provisional application No. 62/327,305, filed on Apr. 25, 2016.

(51) Int. Cl. A61F 9/02

(52) U.S. Cl. CPC A61F 9/025; A61F 9/027

(57) ABSTRACT

A goggle includes a frame, a lens, and a locking system. The frame has a first lateral portion, an opposing second lateral portion, an upper portion, and a lower portion defining a cavity therebetween. The lens is disposed across the cavity. The locking system is configured to selectively secure the lens to the frame. The locking system includes a first locking member releasably coupled to the first lateral portion of the frame and a second locking member releasably coupled to the opposing second lateral portion of the frame.

### CROSS-REFERENCE TO RELATED PATENT APPLICATIONS

This application claims the benefit of U.S. Provisional Patent Application No. 62/327,305, filed Apr. 25, 2016, which is incorporated herein by reference in its entirety.

### BACKGROUND

The subject matter disclosed herein generally relates to an attachment system (e.g., coupling system, outrigger locking system, a quick release system, etc.) configured to couple a lens to and/or within a frame of eyewear (e.g., goggles, glasses, sunglasses, protective eyewear, etc.). Such an attachment system may be used in conjunction with protective helmets or other protective gear configured for sports or vocations.

Eyewear, such as goggles, glasses, sunglasses, etc., often include one or more lenses that are disposed within a frame of the eyewear to hold the lenses in place. Such an arrangement may make it difficult to interchange and/or replace lenses.

### SUMMARY

The subject matter disclosed herein offers solutions to the challenges with interchanging and/or replacing lenses in conventional eyewear.

One embodiment relates to a goggle. The goggle includes a frame, a lens, and a locking system. The frame has a first lateral portion, an opposing second lateral portion, an upper portion, and a lower portion defining a cavity therebetween. The lens is disposed across the cavity. The locking system is configured to selectively secure the lens to the frame. The locking system includes a first locking member releasably coupled to the first lateral portion of the frame and a second locking member releasably coupled to the opposing second lateral portion of the frame.

Another embodiment relates to an attachment system configured to selectively secure a lens to a frame of an eyewear. The attachment system includes a first attachment member and a second attachment member. The first attachment member is configured to releasably couple to a first lateral portion of the frame. The first attachment member includes a first engagement portion configured to interface with a first coupler extending from the first lateral portion of the frame to selectively secure a first end of the lens between the first lateral portion and the first engagement portion. The second attachment member is configured to releasably couple to an opposing second lateral portion of the frame. The second attachment member includes a second engagement portion configured to interface with a second coupler extending from the opposing second lateral portion of the frame to selectively secure an opposing second end of the lens between the opposing second lateral portion and the second engagement portion.

Still another embodiment relates an eyewear. The eyewear includes a frame, a lens, and an attachment system. The frame has a first lateral portion including a first coupler and an opposing second lateral portion including a second coupler. The lens has a first end defining a first aperture and an opposing second end defining a second aperture. The first aperture and the second aperture are positioned to receive the first coupler and the second coupler, respectively. The attachment system is configured to selectively secure the lens to the frame. The attachment system includes a first attachment member and a second attachment member. The first attachment member includes a first engagement portion configured to interface with the first coupler such that the first coupler extends through the first aperture of the lens and the first engagement portion of the first attachment member, thereby selectively coupling the first attachment member to the first lateral portion of the frame and the first end of the lens between the first lateral portion of the frame and the first engagement portion of the first attachment member. The second attachment member includes a second engagement portion configured to interface with the second coupler such that the second coupler extends through the second aperture of the lens and the second engagement portion of the second attachment member, thereby selectively coupling the second attachment member to the opposing second lateral portion of the frame and the opposing second end of the lens between the opposing second lateral portion of the frame and the second engagement portion of the second attachment member.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The drawings are provided to illustrate example embodiments described herein and are not intended to limit the scope of the disclosure. Throughout the drawings, reference numbers may be re-used to indicate general correspondence between referenced elements.

FIG. 1 is a front perspective view of eyewear, according to an exemplary embodiment;

FIG. 2 is an exploded, perspective view of the eyewear of FIG. 1, according to an exemplary embodiment;

FIG. 3 is a front view of the eyewear of FIG. 1, according to an exemplary embodiment;

FIG. 4 is a rear perspective view of a right end of the eyewear of FIG. 1, according to an exemplary embodiment;

FIG. 5 is a rear perspective view of a left end of the eyewear of FIG. 1, according to an exemplary embodiment;

FIG. 6 is a front view of eyewear, according to another exemplary embodiment;

FIG. 7 is a rear perspective view of the eyewear of FIG. 6, according to an exemplary embodiment;

FIG. 8 is a right side view of the eyewear of FIG. 6, according to an exemplary embodiment;

FIG. 9 is a rear perspective view of a right end of the eyewear of FIG. 6, according to an exemplary embodiment;

FIGS. 10 and 11 are various perspective views of a frame of eyewear, according to an exemplary embodiment;

FIGS. 12A and 12B are various perspective views of a lens of eyewear, according to an exemplary embodiment;

FIG. 13 is a perspective view of a lens of eyewear, according to another exemplary embodiment;

FIGS. 14A-18B are various detailed views of an attachment system of eyewear, according to an exemplary embodiment; and

FIG. 19 is a detailed view a head strap of eyewear, according to an exemplary embodiment.

### DETAILED DESCRIPTION

Various aspects of the disclosure will now be described with regard to certain examples and embodiments, which are intended to illustrate but not to limit the disclosure. Nothing in this disclosure is intended to imply that any particular feature or characteristic of the disclosed embodiments is essential. The scope of protection is defined by the claims that follow this description and not by any particular embodiment described herein. Before turning to the figures, which illustrate example embodiments in detail, it should be understood that the application is not limited to the details or methodology set forth in the description or illustrated in the figures. It should also be understood that the terminology is for the purpose of description only and should not be regarded as limiting.

Embodiments herein generally relate to an attachment system (e.g., coupling system, outrigger locking system, a quick release system, etc.) configured to couple a lens to and/or within a frame of eyewear (e.g., goggles, glasses, sunglasses, protective eyewear, etc.). The attachment system may be configured to facilitate selectively and/or releasably coupling the lens to the frame in a relatively simple (e.g., easy, quick, etc.) and/or toolless manner (e.g., does not require the use of tools such as a screw driver, etc.). Such eyewear may be used in a number of activities, including without limitation: sports and athletics, including extreme sports such as motocross and snowmobiling; cycling activities, including auto racing, motorcycle riding and racing, BMX, mountain biking, etc.; with recreational vehicles including all-terrain vehicles (ATVs), dirt bikes, utility task vehicles (UTVs), snowmobiles, and other off-road vehicles; military applications; and/or construction applications to name just a few. Further details are provided herein.

According to an exemplary embodiment, the eyewear of the present disclosure includes a frame, a lens, and an attachment system. The frame has a first lateral portion (e.g., a left end, a right end, etc.), an opposing second lateral portion (e.g., a right end, a left end, etc.), an upper portion, and a lower portion, all of which cooperatively define a cavity or recess therebetween. The first lateral portion of the frame includes a first coupler (e.g., a fastener, locking pins, a pair of couplers, etc.) extending therefrom (e.g., integrally formed with, coupled to, etc.). The opposing second lateral portion includes a second coupler (e.g., a fastener, locking pins, a pair of couplers, etc.) extending therefrom (e.g., integrally formed with, coupled to, etc.). The upper portion of the frame may define at least one of a first engagement surface and a first recess positioned towards the first lateral portion of the frame (e.g., on the left of a central, vertical axis of the frame; on the right of a central, vertical axis of the frame; etc.). The upper portion of the frame may further define at least one of a second engagement surface and a second recess positioned towards the opposing second lateral portion of the frame (e.g., on the right of a central, vertical axis of the frame; on the left of a central, vertical axis of the frame; etc.).

According to an exemplary embodiment, the lens is disposed and/or extends across (e.g., supported by, mounted therein, etc.) the cavity or recess defined by the frame. The lens may have a first end (e.g., a left end, a right end, a left flange, a right flange, etc.) shaped to correspond with the first lateral portion of the frame. The first end of the lens may define a first interface (e.g., a pair of apertures, etc.) positioned to correspond with the first coupler such that the first coupler extends therethrough. The lens may also have an opposing second end (e.g., a right end, a left end, a right flange, a left flange, etc.) shaped to correspond with the second lateral portion of the frame. The opposing second end of the lens may define a second interface (e.g., a pair of apertures, etc.) positioned to correspond with the second coupler such that the second coupler extends therethrough.

The attachment system is configured to selectively and/or releasably secure the lens to the frame (e.g., in a toolless manner, a quick-release manner, etc.). The attachment system includes a first attachment member and a second attachment member. The first attachment member includes a first engagement portion configured to interface with the first coupler. The first coupler may be received by and extend through the first engagement portion of the first attachment member and the first aperture of the lens to thereby selectively couple the first attachment member to the first lateral portion of the frame and selectively secure the first end of the lens between the first lateral portion of the frame and the first engagement portion of the first attachment member. The second attachment member includes a second engagement portion configured to interface with the second coupler. The second coupler may be received by and extend through the second engagement portion of the second attachment member and the second aperture to thereby selectively couple the second attachment member to the second lateral portion of the frame and to selectively secure the opposing second end of the lens between the opposing second lateral portion of the frame and the second engagement portion of the second attachment member.

The first attachment member may include a first arm extending from the first engagement portion and shaped to correspond with the first engagement surface of the upper portion of the frame. According to an exemplary embodiment, the first engagement portion and the first arm are integrally formed such that the first attachment member has a single, continuous (i.e. unitary) structure. The first arm may include a first locking portion positioned along the first arm (e.g., at an end thereof, etc.). The first locking portion may include at least one of (i) a first frame engagement portion (e.g., a first latch, etc.) configured to correspond with the first recess defined by the upper portion of the frame and (ii) a first lens engagement portion configured to extend downward and along a front surface of the lens.

The second attachment member may include a second arm extending from the second engagement portion and shaped to correspond with the second engagement surface of the upper portion of the frame. According to an exemplary embodiment, the second engagement portion and the second arm are integrally formed such that the second attachment member has a single, continuous (i.e., unitary) structure. The second arm may include a second locking portion positioned along the second arm (e.g., at an end thereof, etc.). The second locking portion may include at least one of (i) a second frame engagement portion (e.g., a second latch, etc.) configured to correspond with the second recess defined by the upper portion of the frame and (ii) a second lens engagement portion configured to extend downward and along the front surface of the lens. The first lens engagement portion and the second lens engagement portion may thereby cooperatively hold a top edge of the lens against the upper portion of the frame.

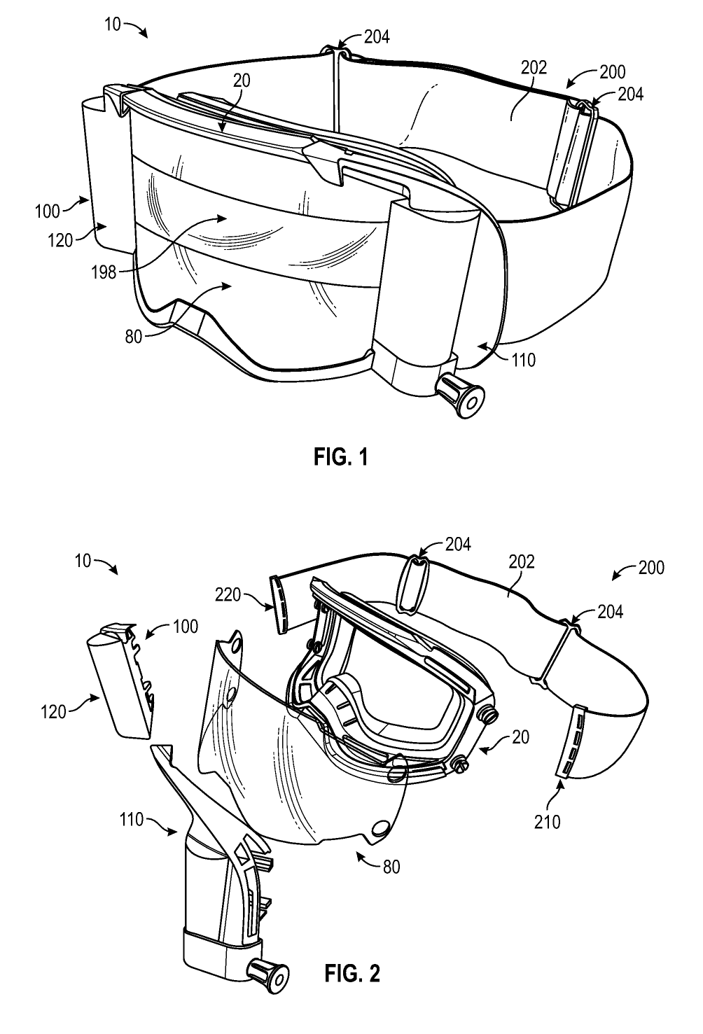

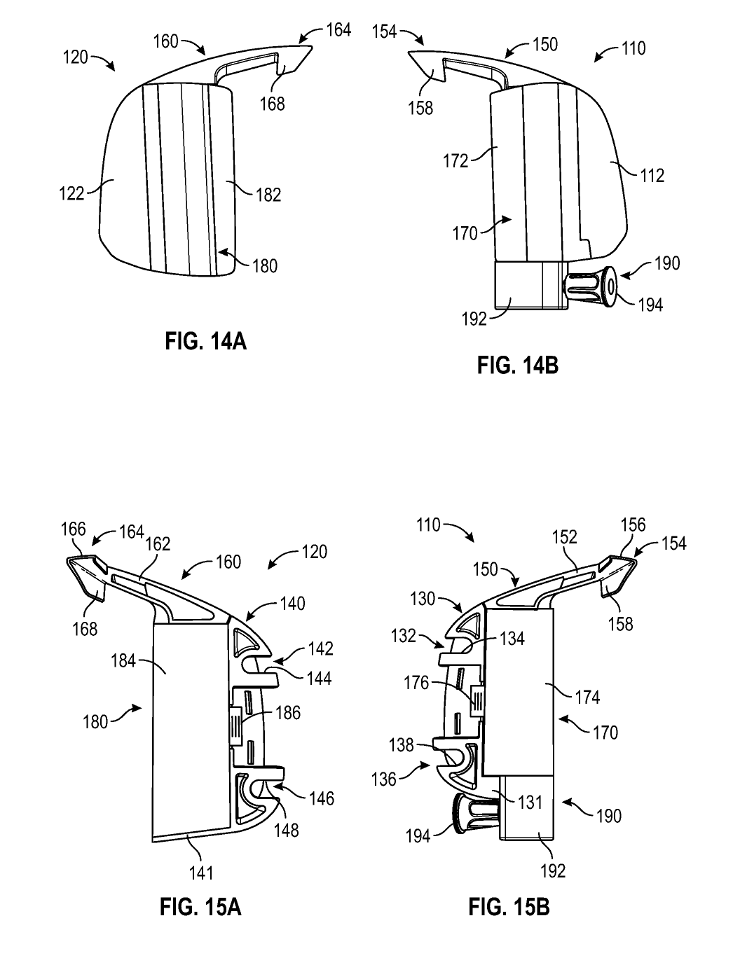

According to the exemplary embodiment shown in FIGS. 1-19, an eyewear, shown as goggles 10, include a frame, shown as frame 20, a lens, shown as lens 80, an attachment system, shown as outrigger locking system 100, and a strap, shown as head strap 200. According to an exemplary embodiment, the outrigger locking system 100 is configured to selectively and/or releasably secure (e.g., attach, couple, etc.) the lens 80 to the frame 20 of the goggles 10. In an alternative embodiment, the outrigger locking system 100 is configured to selectively and/or releasably secure the lens 80 (e.g., a visor, etc.) to a helmet (e.g., a football helmet, a hockey helmet, a lacrosse helmet, a welding helmet, a motorcycle helmet, etc.). According to an exemplary embodiment, the eyewear are motocross goggles. In other embodiments, the eyewear are snowmobiling goggles, snowboarding goggles, mountain biking goggles, motorcycle goggles, sky diving goggles, or still another action or extreme sport goggles. In still other embodiments, the eyewear are swimming goggles or other sports goggles (e.g., used in basketball, baseball, etc.; Rec Specs®; etc.). In yet other embodiments, the eyewear are another type of eyewear used for construction, military applications, machining, carpentry, scientific experimentation, and/or the like. In still other embodiments, the eyewear are traditional vision enhancing glasses (e.g., prescription glasses, etc.) and/or sunglasses.

According to an exemplary embodiment, the goggles 10 provide protection to the eyes and adjacent area of the face of a wearer. The lens 80 and/or frame 20 may intercept light, wind, rain, snow, water, particulate matter (e.g., dust, dirt, mud, etc.), and the like to protect the eyes and/or various portions of the face of a wearer of the goggles 10.

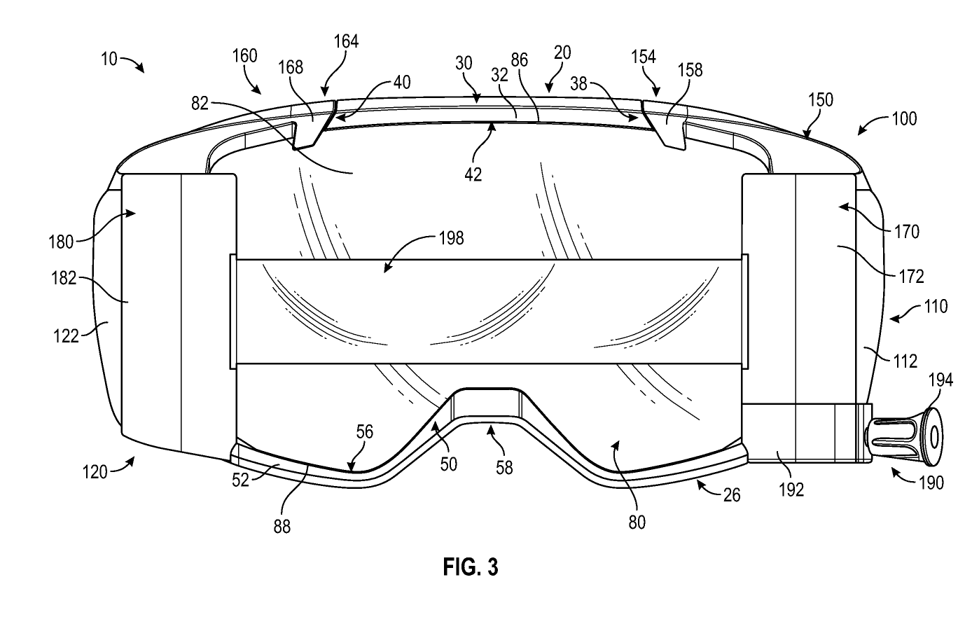

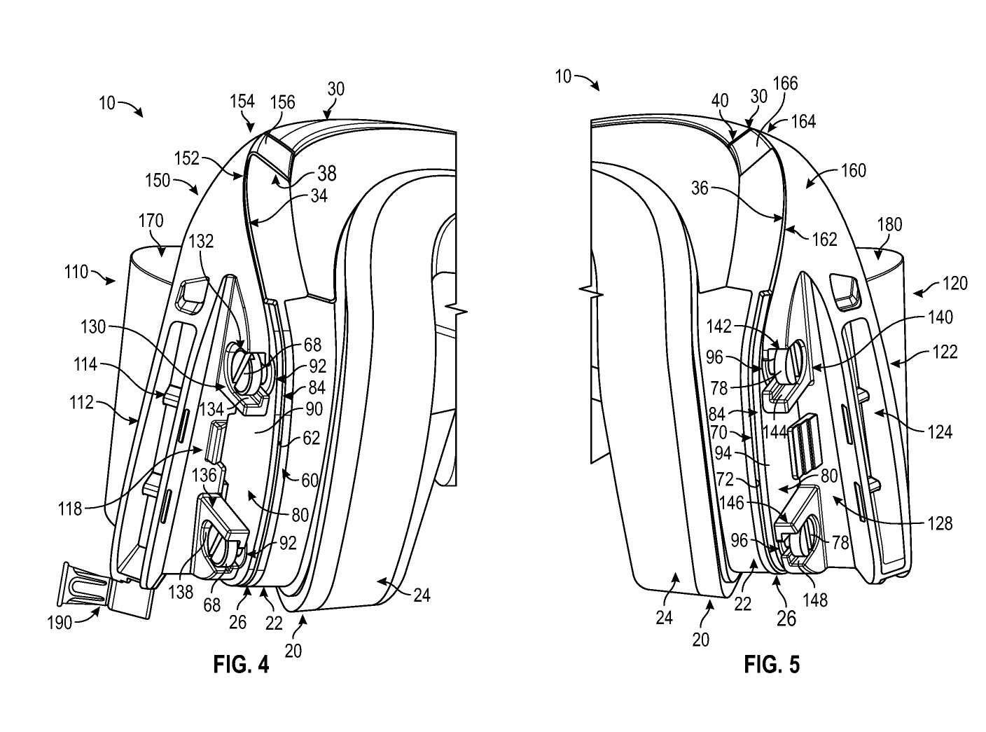

As shown in FIGS. 3-11, the frame 20 includes a first frame portion, shown as inner frame 22, a cushion member, shown as compressible member 24, and second frame portion, shown as outer frame 26. According to an example embodiment, the inner frame 22 is shaped to correspond with anatomical features of a wearer's face (e.g., cheeks, nose, forehead, etc.) to facilitate a proper fit of the goggles 10 when worn. As shown in FIGS. 4,5, 10, and 11, the compressible member 24 is positioned along an interior of the inner frame 22 such that the compressible member 24 is in contact with a wearer's face when the goggles 10 are worn. The compressible member 24 may include an impact attenuating material and/or cushion material (e.g., expanded polyurethane (EPU) foam, expanded polypropylene (EPP) foam, expanded polyethylene (EPE) foam, polyolefin foam, etc.) to facilitate a snug fit and comfortable fit of the goggles 10 onto a wearer's face and/or mitigate impact forces encountered by the goggles 10 (e.g., from flying debris, etc.). As shown in FIGS. 4-8, 10, and 11, the outer frame 26 is positioned along an exterior of the inner frame 22. According to an exemplary embodiment, the outer frame 26 is configured to facilitate coupling the lens 80 to the frame 20 via the outrigger locking system 100.

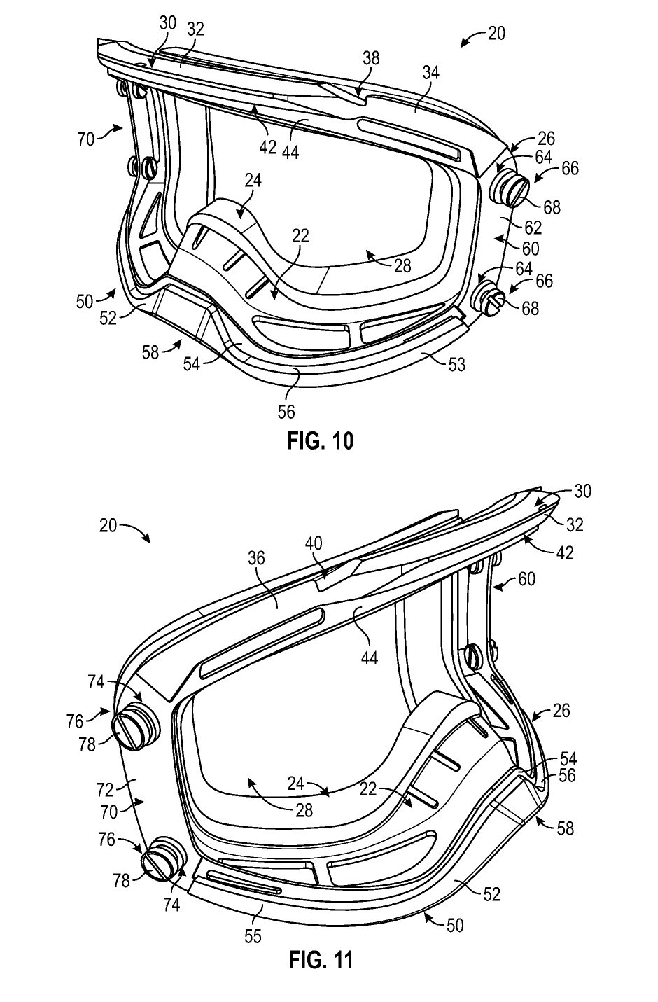

As shown in FIGS. 3-11, the outer frame 26 has an upper portion, shown as upper frame portion 30, a lower portion, shown as lower frame portion 50, a first lateral portion, shown as right frame end 60, an opposing second lateral portion, shown as left frame end 70. The upper frame portion 30, the lower frame portion 50, the right frame end 60, and the left frame end 70 cooperatively define a cavity or recess therebetween, shown as lens opening 28. As shown in FIGS. 4 and 7-10, the right frame end 60 of the outer frame 26 has a first face, shown as right lens surface 62. According to the exemplary embodiment shown in FIG. 10, the right lens surface 62 defines a plurality of first apertures (e.g., two, three, four, etc.), shown as right coupling apertures 64. The right coupling apertures 64 are configured (e.g., positioned, sized, etc.) to receive a corresponding number of first coupling members (e.g., two, three, four, etc.), shown as right couplers 66, such that the right couplers 66 extend from the right lens surface 62. In other embodiments, the right lens surface 62 defines a single right coupling aperture 64. In other embodiments, the right couplers 66 are integrally formed with and extend from the right lens surface 62. According to the exemplary embodiment shown in FIG. 10, the right couplers 66 include first pins, shown as right locking pins 68. In some embodiments, some or all of the right locking pins 68 include a keyed feature. In other embodiments, the right couplers 66 include another type of coupler and/or fastener (e.g., magnets, hook and loop, a clip, a latch, screws, bolts, etc.).

As shown in FIGS. 5 and 11, the left frame end 70 of the outer frame 26 has a second face, shown as left lens surface 72. According to the exemplary embodiment shown in FIG. 11, the left lens surface 72 defines a plurality of second apertures (e.g., two, three, four, etc.), shown as left coupling apertures 74. The left coupling apertures 74 are configured (e.g., positioned, sized, etc.) to receive a corresponding number of second coupling members (e.g., two, three, four, etc.), shown as left couplers 76, such that the left couplers 76 extend from the left lens surface 72. In other embodiments, the left lens surface 72 defines a single left coupling aperture 74. In other embodiments, the left couplers 76 are integrally formed with and extend from the left lens surface 72. According to the exemplary embodiment shown in FIG. 11, the left couplers 76 include second pins, shown as left locking pins 78. In some embodiments, some or all of the left locking pins 78 include a keyed feature. In other embodiments, the left couplers 76 include another type of coupler and/or fastener (e.g., magnets, hook and loop, a clip, a latch, screws, bolts, etc.).

As shown in FIGS. 3, 6, 8, 10, and 11, the upper frame portion 30 of the outer frame 26 includes an upper extension, shown as upper brim 32, extending from an upper surface of the outer frame 26, shown as upper lens interface 44. As shown in FIGS. 4, 7, 8, and 10, the upper brim 32 defines a first engagement surface, shown as right, upper outrigger engagement surface 34. As shown in FIGS. 3-4, 6-8, and 10, the upper brim 32 defines a first recess, shown as right locking interface 38. According to an exemplary embodiment, the right, upper outrigger engagement surface 34 and the right locking interface 38 are positioned towards the right frame end 60 of the outer frame 26 (e.g., on the right side of a central, vertical axis of the frame 20; etc.). As shown in FIGS. 5, 7, and 11, the upper brim 32 defines a second engagement surface, shown as left, upper outrigger engagement surface 36. As shown in FIGS. 3, 5-7, and 11, the upper brim 32 defines a second recess, shown as left locking interface 40. According to an exemplary embodiment, the left, upper outrigger engagement surface 36 and the left locking interface 40 are positioned towards the left frame end 70 of the outer frame 26 (e.g., on the left side of a central, vertical axis of the frame 20; etc.). As shown in FIGS. 3, 6, 8, 10, and 11, the upper brim 32 defines an underside or lower ledge, shown as upper lip 42.

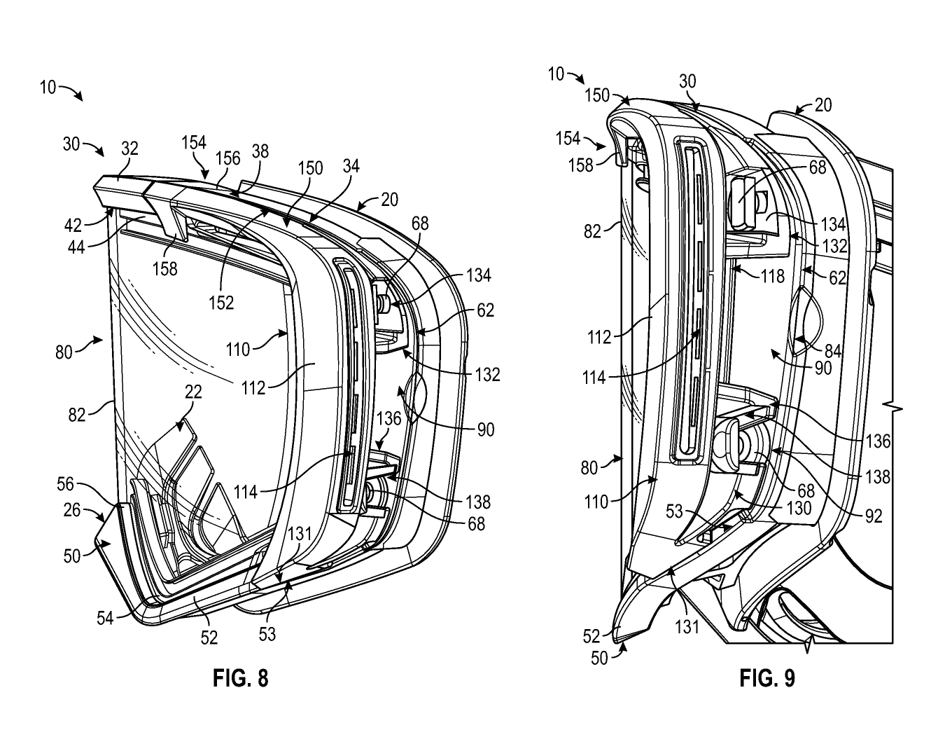

As shown in FIGS. 3, 6, 8, 10, and 11, the lower frame portion 50 of the outer frame 26 includes a lower extension, shown as lower brim 52, extending from a lower surface of the outer frame 26, shown as lower lens interface 54. As shown in FIGS. 8-10, the lower brim 52 defines a first engagement surface, shown as right, lower outrigger engagement surface 53, positioned towards the right frame end 60 of the outer frame 26 (e.g., on the right side of a central, vertical axis of the frame 20; etc.). As shown in FIG. 11, the lower brim 52 defines a second engagement surface, shown as left, lower outrigger engagement surface 55, positioned towards the left frame end 70 of the outer frame 26 (e.g., on the left side of a central, vertical axis of the frame 20; etc.). As shown in FIGS. 3, 6, 8, 10, and 11, the lower brim 52 defines an upper side or upper edge, shown as lower lip 56. As shown in FIGS. 3, 6, 7, 10, and 11, the lower frame portion 50 defines an indent, shown as nose opening 58, shaped to correspond with the nose of a wearer of the goggles 10.

According to the exemplary embodiment shown in FIGS. 1-3, 6, 7, and 12A-13, the lens 80 includes a unitary, arcuate lens that extends across the entire lens opening 28 (e.g., both the left and right eyes of the wearer, etc.). In an alternative embodiment, the lens 80 includes dual lenses, one positioned over each of the left and right eyes of the wearer. In some embodiments, the lens 80 is clear (e.g., substantially transparent, etc.). In other embodiments, the lens 80 includes a tinted and/or polarized coating to shade a wearer's eyes from sunlight, reduce glare, improve contrast, and/or enhance depth perception. The tinted and/or polarized coating may be various colors (e.g., black, yellow, blue, green, brown, gray, red, etc.). In some embodiments, the lens 80 includes a reflective coating (e.g., to prevent others from seeing where the wearer is looking, etc.). In some embodiments, the lens 80 is a prescription lens configured to enhance the vision of a wearer of the goggles 10.

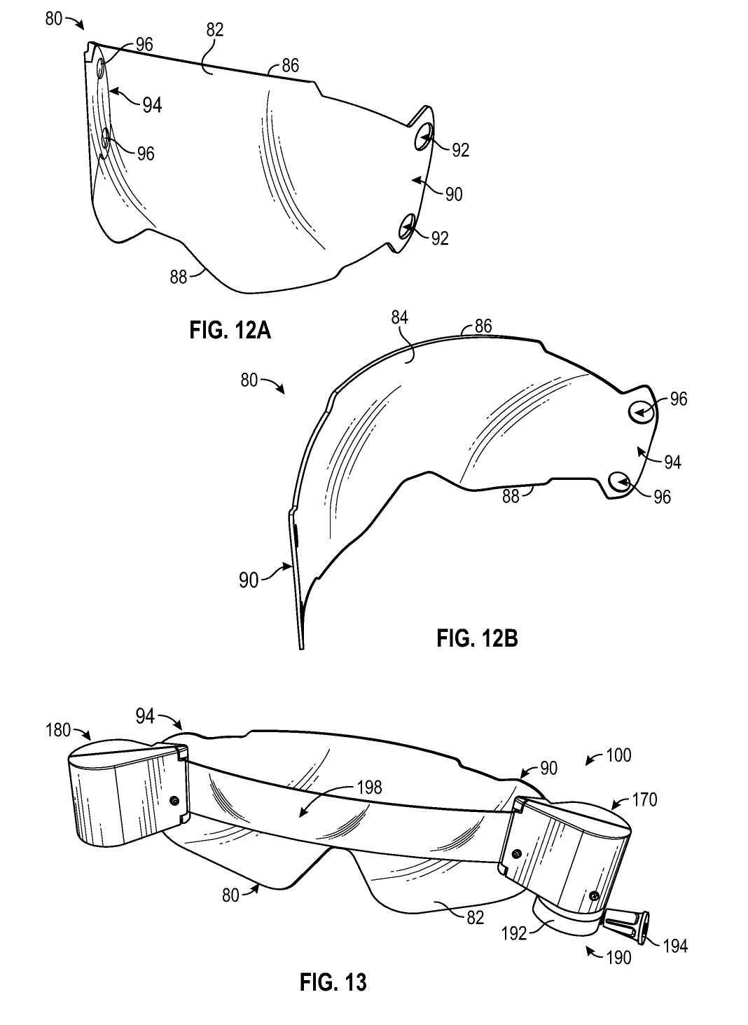

As shown in FIGS. 3, 6-9, and 12A-13, the lens 80 has a first side, shown as front surface 82, an opposing second side, shown as rear surface 84, a first edge, shown as upper edge 86, an opposing second edge, shown as lower edge 88, a first end, shown as right flange 90, and an opposing second end, shown as left flange 94. According to an exemplary embodiment, the right flange 90 is shaped to correspond with the right lens surface 62 of the right frame end 60 of the outer frame 26 and the left flange 94 is shaped to correspond with the left lens surface 72 of the left frame end 70 of the outer frame 26. As shown in FIG. 12A, the right flange 90 defines a first plurality of apertures, shown as right apertures 92, to correspond with the right couplers 66 of the frame 20. As shown in FIGS. 12A and 12B, the left flange 94 defines a second pair of apertures, shown as left apertures 96, to correspond with the left couplers 76 of the frame 20.

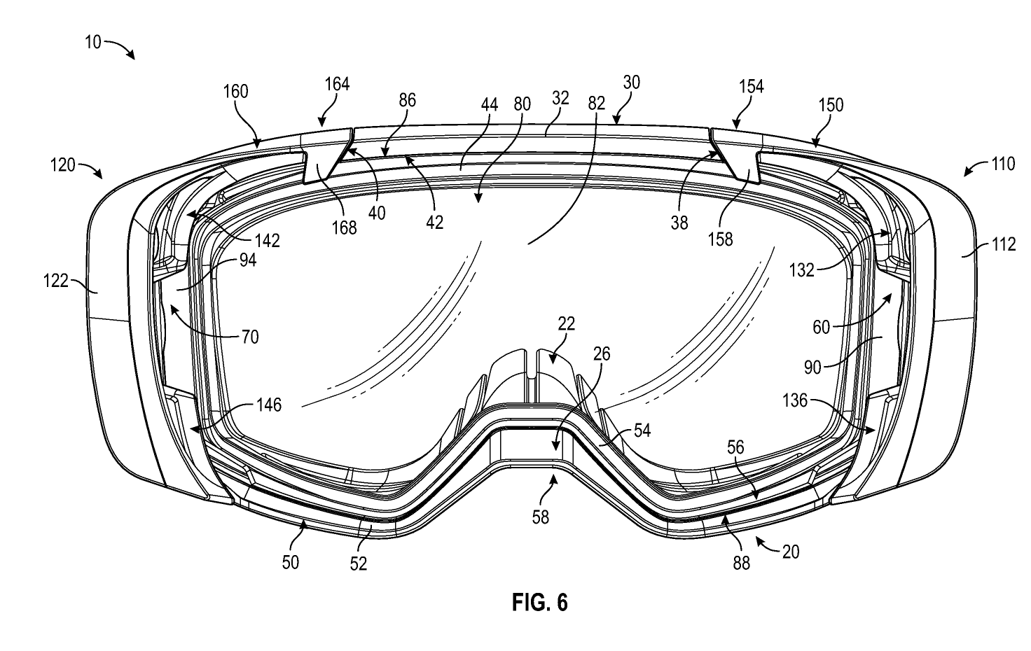

As shown in FIGS. 1 and 3-9, the lens 80 is disposed across and/or along, and supported by the frame 20. As shown in FIGS. 3, 6, and 8, the rear surface 84 and the upper edge 86 of the lens 80 are disposed along and supported by the upper lip 42 and the upper lens interface 44 of the upper frame portion 30. As shown in FIGS. 3, 6, and 8, the rear surface 84 and the lower edge 88 of the lens 80 are disposed along and supported by the lower lip 56 and the lower lens interface 54 of the lower frame portion 50. As shown in FIGS. 4, 6, 8, and 9, the right flange 90 of the lens 80 is disposed along the right lens surface 62 of the right frame end 60 of the outer frame 26. As shown in FIGS. 4 and 9, the right apertures 92 defined by the right flange 90 are configured (e.g., positioned, sized, etc.) to receive the right couplers 66 such that the right locking pins 68 extend therethrough. As shown in FIGS. 5 and 6, the left flange 94 of the lens 80 is disposed along the left lens surface 72 of the left frame end 70 of the outer frame 26. As shown in FIG. 5, the left apertures 96 defined by the left flange 94 are configured (e.g., positioned, sized, etc.) to receive the left couplers 76 such that the left locking pins 78 extend therethrough. Thus, the upper lip 42, the upper lens interface 44, the lower lip 56, the lower lens interface 54, the right lens surface 62, the right couplers 66, the left lens surface 72, and/or the left couplers 76 may support the lens 80 such that the lens 80 is selectively secured to the outer frame 26 (e.g., prevented from moving left to right and top to bottom, etc.).

As shown in FIGS. 1-3, the outrigger locking system 100 includes a first attachment or locking member, shown as right outrigger 110, and a second attachment or locking member, shown as left outrigger 120. According to an exemplary embodiment, the right outrigger 110 and the left outrigger 120 are configured to releasably couple to right frame end 60 and the left frame end 70, respectively, to selectively secure the right flange 90 and the left flange 94 of the lens 80 to the outer frame 26 (e.g., to prevent the lens 80 from disengaging from the right couplers 66 and the left couplers 76, etc.).

As shown in FIGS. 3, 4, 6-9, 14B, 15B, 16B, 17B, and 18B, the right outrigger 110 includes a first portion, shown as right strap engagement portion 112, a second portion, shown as right coupler engagement portion 130, and a third portion, shown as right arm 150. As shown in FIGS. 3, 5-7, 14A, 15A, 16A, 17A, and 18A, the left outrigger 120 includes a first portion, shown as left strap engagement portion 122, a second portion, shown as left coupler engagement portion 140, and a third portion, shown as left arm 160. As shown in FIGS. 3-5 and 14A-18B, the right outrigger 110 further includes a first canister, shown as intake canister 170, and an actuation system, shown as roll-off actuation system 190, and the left outrigger 120 includes a second canister, shown as supply canister 180. According to an exemplary embodiment, the right outrigger 110 (e.g., the right strap engagement portion 112, the right coupler engagement portion 130, the right arm 150, the intake canister 170, and/or the roll-off actuation system 190, etc.) and/or the left outrigger 120 (e.g., the left strap engagement portion 122, the left coupler engagement portion 140, the left arm 160, and/or the supply canister 180, etc.) have a unitary structure (e.g., integrally formed; a single, continuous component; etc.).

As shown in FIGS. 4, 7-9, 15B, 16B, and 18B, the right coupler engagement portion 130 of the right outrigger 110 includes a first interface, shown as upper interface 132, and a second interface, shown as lower interface 136. The upper interface 132 defines a first coupling portion, shown as upper coupler 134, and the lower interface 136 defines a second coupling portion, shown as lower coupler 138. In other embodiments, the right coupler engagement portion 130 includes a different number of interfaces (e.g., one, three, to correspond with the number of right couplers 66, etc.). As shown in FIGS. 4 and 7-9, the upper interface 132 and the lower interface 136 of the right coupler engagement portion 130 are configured (e.g., positioned, sized, structured, etc.) to interface with the right couplers 66. One of the right locking pins 68 may be received by and/or extend through each of the upper coupler 134, the lower coupler 138, and the right apertures 92 of the lens 80, thereby selectively coupling the right outrigger 110 to the right frame end 60 of the frame 20, as well as selectively securing the right flange 90 of the lens 80 between the right lens surface 62 of the right frame end 60 of the outer frame 26 and the right coupler engagement portion 130 of the right outrigger 110.

According to the exemplary embodiment shown in FIGS. 4, 15B, 16B, and 18B, the upper coupler 134 and the lower coupler 138 of the right outrigger 110 are structured as slots configured to slidably engage with the right locking pins 68 to releasably couple the right outrigger 110 to the outer frame 26 of the goggles 10. According to the exemplary embodiment shown in FIGS. 8 and 9, the upper coupler 134 is structured as an aperture, and the lower coupler 138 is structured as a slot. According to an exemplary embodiment, the aperture of the upper coupler 134 is shaped to correspond with a keyed feature of a corresponding, upper right locking pin 68. The upper right locking pin 68 may thereby be received by and extend through the aperture of the upper coupler 134 and then rotated to prevent the upper interface 132 of the right outrigger 110 from disengaging from the upper right locking pin 68. The aperture of the upper interface 132 and the slot of the lower interface 136 may thereby facilitate pivotably coupling the right outrigger 110 to the outer frame 26 such that the right outrigger 110 pivots about the upper right locking pin 68. In other embodiments, the upper coupler 134 and/or the lower coupler 138 are otherwise structured (e.g., include an aperture, a slot, a clip, an interface, a latch, a magnet, a magnetically attractive material, etc. to correspond with the right couplers 66 of the frame 20, etc.) to otherwise releasably couple the right outrigger 110 to the frame 20 (e.g., snap fit, twist fit, magnetically, etc.).

As shown in FIGS. 5, 15A, 16A, and 18A, the left coupler engagement portion 140 of the left outrigger 120 includes a first interface, shown as upper interface 142, and a second interface, shown as lower interface 146. The upper interface 142 defines a first coupling portion, shown as upper coupler 144, and the lower interface 146 defines a second coupling portion, shown as lower coupler 148. In other embodiments, the left coupler engagement portion 140 includes a different number of interfaces (e.g., one, three, to correspond with the number of left couplers 76, etc.). As shown in FIG. 5, the upper interface 142 and the lower interface 146 of the left coupler engagement portion 140 are configured (e.g., positioned, sized, structured, etc.) to interface with the left couplers 76. One of the left locking pins 78 may be received by and/or extend through each of the upper coupler 144, the lower coupler 148, and the left apertures 96 of the lens 80, thereby selectively coupling the left outrigger 120 to the left frame end 70 of the frame 20, as well as selectively securing the left flange 94 of the lens 80 between the left lens surface 72 of the left frame end 70 of the outer frame 26 and the left coupler engagement portion 140 of the left outrigger 120.

According to the exemplary embodiment shown in FIGS. 5, 15A, 16A, and 18A, the upper coupler 144 and the lower coupler 148 of the left outrigger 120 are structured as slots configured to slidably engage with the left locking pins 78 to releasably couple the left outrigger 120 to the outer frame 26 of the goggles 10. According to another exemplary embodiment, the upper coupler 144 is structured as an aperture, and the lower coupler 138 is structured as a slot. The aperture of the upper coupler 144 may be shaped to correspond with a keyed feature of a corresponding, upper left locking pin 78. The upper left locking pin 78 may thereby be received by and extend through the aperture of the upper coupler 144 and then rotated to prevent the upper interface 142 of the left outrigger 120 from disengaging from the upper left locking pin 78. The aperture of the upper interface 142 and the slot of the lower interface 146 may thereby facilitate pivotably coupling the left outrigger 120 to the outer frame 26 such that the left outrigger 120 pivots about the upper left locking pin 78. In other embodiments, the upper coupler 144 and/or the lower coupler 148 are otherwise structured (e.g., include an aperture, a slot, a clip, an interface, a latch, a magnet, a magnetically attractive material, etc. to correspond with the left couplers 76 of the frame 20, etc.) to otherwise releasably couple the left outrigger 120 to the frame 20 (e.g., snap fit, twist fit, magnetically, etc.).

As shown in FIGS. 3, 4, 6-9, 14B, 15B, 16B, and 18B, the right arm 150 extends from an upper portion of the right strap engagement portion 112 and/or the right coupler engagement portion 130. As shown in FIGS. 4, 7, 8, 15B, 16B, and 18B, the right arm 150 defines a rear surface, shown as upper frame engagement surface 152. As shown in FIGS. 4, 7, and 8, the upper frame engagement surface 152 of the right arm 150 is shaped to correspond with and be disposed along the right, upper outrigger engagement surface 34 of the upper frame portion 30.

As shown in FIGS. 3, 4, 6-9, 14B, 15B, 16B, 17B, and 18B, the right arm 150 includes a first securing member, shown as right locking member 154, positioned along the right arm 150 (e.g., at an end thereof, etc.). The right locking member 154 includes a first frame engagement portion, shown as right frame latch 156, and a first lens engagement portion, shown as right lens engagement portion 158. As shown in FIGS. 4, 7, and 8, the right frame latch 156 interfaces with (e.g., is received by, etc.) the right locking interface 38 of the upper frame portion 30 (e.g., to further secure the right outrigger 110 to the outer frame 26, etc.). In some embodiments, the right frame latch 156 releasably locks or attaches to and/or within the right locking interface 38 (e.g., magnetically, snap-fit, hook and loop, with a fastener, etc.). As shown in FIGS. 3, 6, 8, and 9, the right lens engagement portion 158 extends downward and along the front surface 82 of the lens 80. According to an exemplary embodiment, the right lens engagement portion 158 is configured to assist in holding (e.g., pressing against, retaining, etc.) the upper edge 86 of the lens 80 against the upper lens interface 44 of the upper frame portion 30. In some embodiments, the right lens engagement portion 158 includes an extension or tab that fits into a corresponding recess or aperture defined by the lens 80 (e.g., such that the right lens engagement portion 158 snaps into place, interlocks with the lens 80, etc.).

As shown in FIGS. 8, 9, 15B, and 16B, the right coupler engagement portion 130 defines a surface, shown as lower frame engagement surface 131. As shown in FIGS. 8 and 9, the lower frame engagement surface 131 is shaped to correspond with and be disposed along the right, lower outrigger engagement surface 53 of the lower frame portion 50. In some embodiments, the right outrigger 110 additionally or alternatively includes a lower, right arm (e.g., similar to the right arm 150, etc.) and/or a lower, right locking member (e.g., similar to the right locking member 154, etc.) that extend from a lower portion of the right strap engagement portion 112 and/or the right coupler engagement portion 130. The lower, right locking member may include a lower frame latch (e.g., similar to right frame latch 156, etc.) and/or a lower lens engagement portion (e.g., similar the right lens engagement portion 158, etc.). By way of example, the lower, right frame latch may interface with (e.g., be received by, etc.) a lower, right locking interface (e.g., similar to the right locking interface 38, etc.) defined by the lower frame portion 50 (e.g., to further secure the right outrigger 110 to the outer frame 26, etc.). In some embodiments, the lower, right frame latch releasably locks or attaches to and/or within the lower, right locking interface (e.g., magnetically, snap-fit, hook and loop, with a fastener, etc.). By way of another example, the lower, right lens engagement portion may extend upward and along the front surface 82 of the lens 80. The lower, right lens engagement portion may be configured to assist in holding (e.g., pressing against, retaining, etc.) the lower edge 88 of the lens 80 against the lower lens interface 54 of the lower frame portion 50. The lower, right lens engagement portion may include an extension or tab that fits into a corresponding recess or aperture defined by the lens 80 (e.g., such that the lower, right lens engagement portion snaps into place; interlocks with the lens 80; etc.).

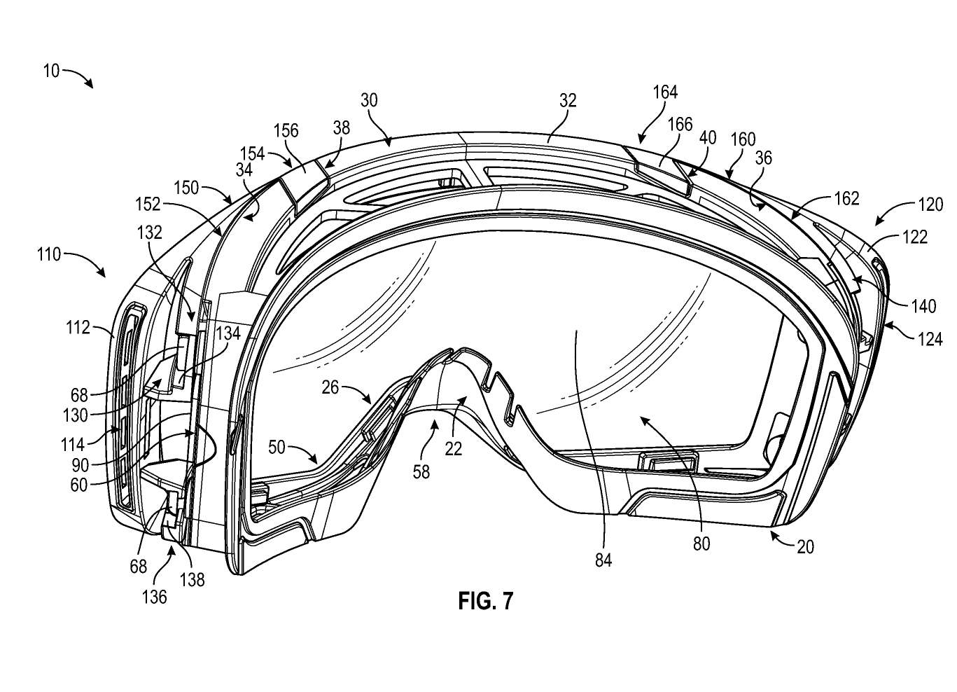

As shown in FIGS. 3, 5-7, 14A, 15A, 16A, and 18A, the left arm 160 extends from an upper portion of the left strap engagement portion 122 and/or the left coupler engagement portion 140. As shown in FIGS. 5, 7, 15A, 16A, and 18A, the left arm 160 defines a rear surface, shown as upper frame engagement surface 162. As shown in FIGS. 5 and 7, the upper frame engagement surface 162 of the left arm 160 is shaped to correspond with and be disposed along the left, upper outrigger engagement surface 36 of the upper frame portion 30.

As shown in FIGS. 3, 5-7, 14A, 15A, 16A, 17A, and 18A, the left arm 160 includes a second securing member, shown as left locking member 164, positioned along the left arm 160 (e.g., at an end thereof, etc.). The left locking member 164 includes a second frame engagement portion, shown as left frame latch 166, and a second lens engagement portion, shown as left lens engagement portion 168. As shown in FIGS. 5 and 7, the left frame latch 166 interfaces with (e.g., is received by, etc.) the left locking interface 40 of the upper frame portion 30 (e.g., to further secure the left outrigger 120 to the outer frame 26, etc.). In some embodiments, the left frame latch 166 releasably locks or attaches to and/or within the left locking interface 40 (e.g., magnetically, snap-fit, hook and loop, with a fastener, etc.). As shown in FIGS. 3 and 6, the left lens engagement portion 168 extends downward and along the front surface 82 of the lens 80. According to an exemplary embodiment, the left lens engagement portion 168 is configured to assist in holding (e.g., pressing against, retaining, etc.) the upper edge 86 of the lens 80 against the upper lens interface 44 of the upper frame portion 30. In some embodiments, the left lens engagement portion 168 includes an extension or tab that fits into a corresponding recess or aperture defined by the lens 80 (e.g., such that the left lens engagement portion 168 snaps into place, interlocks with the lens 80, etc.).

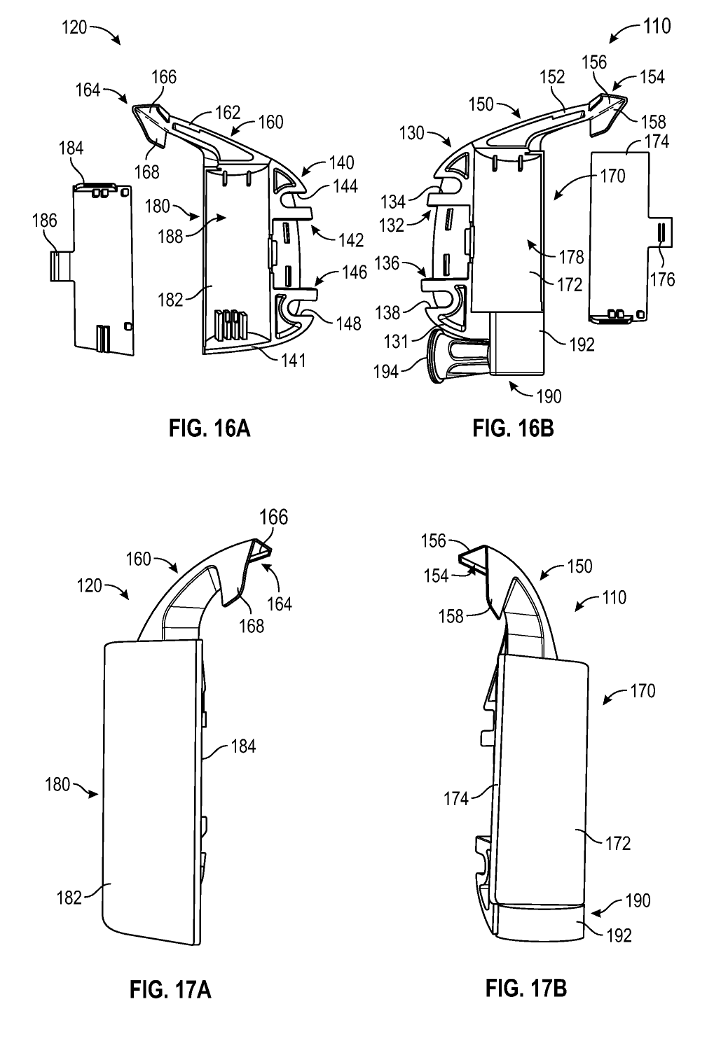

As shown in FIGS. 15A and 16A, the left coupler engagement portion 140 defines a surface, shown as lower frame engagement surface 141. According to an exemplary embodiment, the lower frame engagement surface 141 is shaped to correspond with and be disposed along the left, lower outrigger engagement surface 55 of the lower frame portion 50. In some embodiments, the left outrigger 120 additionally or alternatively includes a lower, left arm (e.g., similar to the left arm 160, etc.) and/or a lower, left locking member (e.g., similar to the left locking member 164, etc.) that extend from a lower portion of the left strap engagement portion 122 and/or the left coupler engagement portion 140. The lower, left locking member may include a lower frame latch (e.g., similar to left frame latch 166, etc.) and/or a lower lens engagement portion (e.g., similar the left lens engagement portion 168, etc.). By way of example, the lower, left frame latch may interface with (e.g., be received by, etc.) a lower, left locking interface (e.g., similar to the left locking interface 40, etc.) defined by the lower frame portion 50 (e.g., to further secure the left outrigger 120 to the outer frame 26, etc.). In some embodiments, the lower, left frame latch releasably locks or attaches to and/or within the lower, left locking interface (e.g., magnetically, snap-fit, hook and loop, with a fastener, etc.). By way of another example, the lower, left lens engagement portion may extend upward and along the front surface 82 of the lens 80. The lower, left lens engagement portion may be configured to assist in holding (e.g., pressing against, retaining, etc.) the lower edge 88 of the lens 80 against the lower lens interface 54 of the lower frame portion 50. The lower, left lens engagement portion may include an extension or tab that fits into a corresponding recess or aperture defined by the lens 80 (e.g., such that the lower, left lens engagement portion snaps into place; interlocks with the lens 80; etc.).

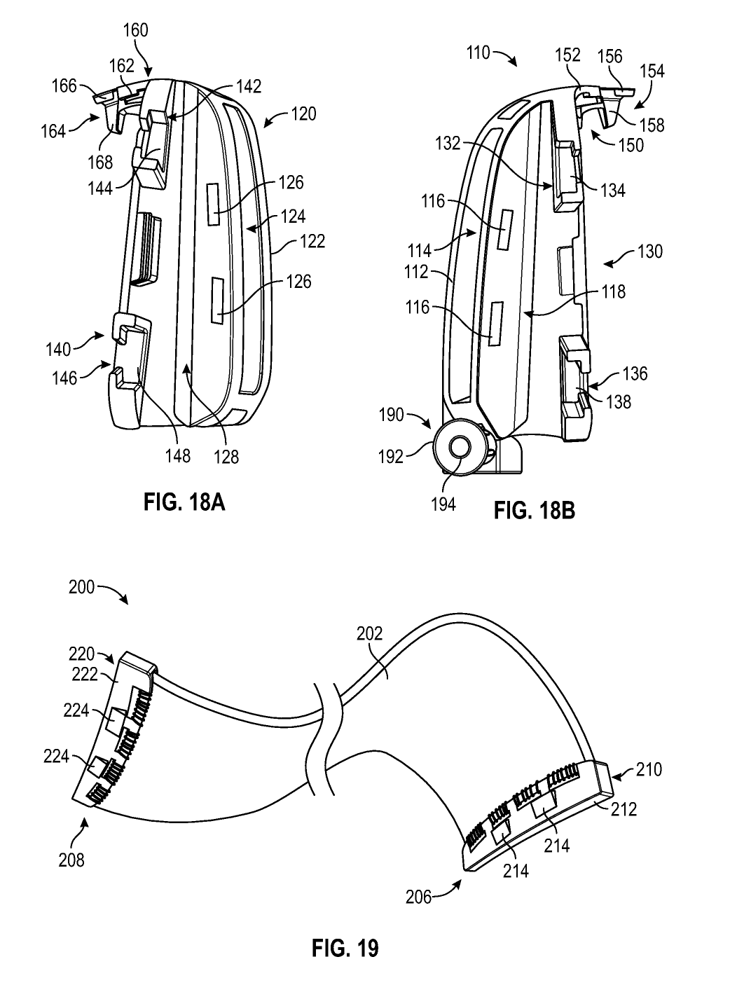

As shown in FIGS. 4, 5, 7-9, 18A, and 18B, the right strap engagement portion 112 of the right outrigger 110 defines a first depression, shown as right recess 114, and the left strap engagement portion 122 of the left outrigger 120 defines a second depression, shown as left recess 124. As shown in FIGS. 18A and 18B, the right strap engagement portion 112 of the right outrigger 110 defines one or more apertures, shown as right securing apertures 116, and the left strap engagement portion 122 of the left outrigger 120 defines one or more apertures, shown as left securing apertures 126.

As shown in FIGS. 1, 2, and 19, the head strap 200 includes a band, shown as head band 202, having a first end, shown as right end 206, and an opposing second end, shown as left end 208. As shown in FIGS. 1 and 2, the head strap 200 includes adjustment members, shown as length adjusters 204. According to an exemplary embodiment, the length adjusters 204 are configured to facilitate adjusting (e.g., extending, shortening, etc.) the length of the head band 202 such that the goggles 10 may fit various sized heads.

As shown in FIGS. 2 and 19, the right end 206 of the head band 202 includes a first connector, shown as right connector 210, and the left end 208 of the head band 202 includes a second connector, shown as left connector 220. As shown in FIG. 19, the right connector 210 includes a body, shown as right clip 212, having one or more retaining elements, shown as right tabs 214, extending therefrom. According to an exemplary embodiment, the right clip 212 is configured to be received by (e.g., shaped to correspond with, etc.) the right recess 114 of the right strap engagement portion 112 of the right outrigger 110. In some embodiments, the right tabs 214 are configured (e.g., shaped, positioned, etc.) to be received by the right securing apertures 116 to selectively and/or releasably secure the right clip 212 within the right recess 114. As shown in FIG. 19, the left connector 220 includes a body, shown as left clip 222, having one or more retaining elements, shown as left tabs 224, extending therefrom. According to an exemplary embodiment, the left clip 222 is configured to be received by (e.g., shaped to correspond with, etc.) the left recess 124 of the left strap engagement portion 122 of the left outrigger 120. In some embodiments, the left tabs 224 are configured (e.g., shaped, positioned, etc.) to be received by the left securing apertures 126 to selectively and/or releasably secure the left clip 222 within the left recess 124.

In some embodiments, the goggles 10 include a roll-off system. As shown in FIGS. 1, 3, and 13, the roll-off system includes the intake canister 170, the supply canister 180, and the roll-off actuation system 190. According to the exemplary embodiment shown in FIGS. 3-5 and 14A-17B, the intake canister 170 is attached to (e.g., integrally formed with, coupled to, etc.) the right outrigger 110, and the supply canister 180 is attached to (e.g., integrally formed with, coupled to, etc.) the left outrigger 120. According to the exemplary embodiment shown in FIG. 13, the intake canister 170 is attached to (e.g., integrally formed with, coupled to, etc.) the front surface 82 of the right flange 90 of the lens 80 and the supply canister 180 is attached to (e.g., integrally formed with, coupled to, etc.) the front surface 82 of the left flange 94 of the lens 80. In other embodiments, the goggles 10 do not include the roll-off system.

As shown in FIGS. 3, 14B, 16B, and 17B, the intake canister 170 includes a front face, shown as intake canister wall 172. According to an exemplary embodiment, the intake canister wall 172 extends outward from the right strap engagement portion 112 and has a half-cylinder shape. In other embodiments, the intake canister wall 172 is otherwise shaped (e.g., square, rectangular, octagon, etc.). As shown in FIGS. 15B, 16B, and 17B, the intake canister 170 includes a cap, shown as rear cap 174, that encloses a cavity, shown as intake roll cavity 178, defined by the intake canister wall 172. As shown in FIGS. 15B and 16B, the rear cap 174 includes a tab, shown as tab 176. According to an exemplary embodiment, the tab 176 is configured to engage a retaining element defined by the intake canister 170 to releasably couple the rear cap 174 to the intake canister 170. As shown in FIGS. 4 and 18B, the right strap engagement portion 112 and the right coupler engagement portion 130 are arranged in a spaced configuration, defining a cavity, shown as cavity 118, therebetween. According to an exemplary embodiment, the cavity 118 facilitates accessing the tab 176 and selectively disengaging the tab 176 from the retaining element to decouple the rear cap 174 from the intake canister 170 (e.g., to access the intake roll cavity 178, etc.).

As shown in FIGS. 3, 14A, 16A, and 17A, the supply canister 180 includes a front face, shown as supply canister wall 182. According to an exemplary embodiment, the supply canister wall 182 extends outward from the left strap engagement portion 122 and has a half-cylinder shape. In other embodiments, the supply canister wall 182 is otherwise shaped (e.g., square, rectangular, octagon, etc.). As shown in FIGS. 15A, 16A, and 17A, the supply canister 180 includes a cap, shown as rear cap 184, that encloses a cavity, shown as supply roll cavity 188, defined by the supply canister wall 182. As shown in FIGS. 15A and 16A, the rear cap 184 includes a tab, shown as tab 186. According to an exemplary embodiment, the tab 186 is configured to engage a retaining element defined by the supply canister 180 to releasably couple the rear cap 184 to the supply canister 180. As shown in FIGS. 5 and 18A, the left strap engagement portion 122 and the left coupler engagement portion 140 are arranged in a spaced configuration, defining a cavity, shown as cavity 128, therebetween. According to an exemplary embodiment, the cavity 128 facilitates accessing the tab 186 and selectively disengaging the tab 186 from the retaining element to decouple the rear cap 184 from the supply canister 180 (e.g., to access the supply roll cavity 188, etc.). In other embodiments, the intake canister wall 172 of the intake canister 170 and/or the supply canister wall 182 of the supply canister 180 are removably coupled to the right outrigger 110 and/or the left outrigger 120, respectively (e.g., with a snap fit, magnetically, such that decoupling the intake canister wall 172 and/or the supply canister wall 182 from the right outrigger 110 and/or the left outrigger 120 exposes the intake roll cavity 178 and/or the supply roll cavity 188, etc.).

According to the exemplary embodiment shown in FIGS. 1, 3, and 13, the roll-off system (e.g., the intake canister 170, the supply canister 180, the roll-off actuation system 190, etc.) is configured to facilitate dispensing a strip of film, shown as film strip 198, across the front surface 82 of the lens 80. According to an exemplary embodiment, the supply roll cavity 188 is configured to store a roll of film and facilitate dispensing the film strip 198 from the roll of film. As shown in FIGS. 3, 4, 13, 14B, 15B, 16B, 17B, and 18B, the roll-off actuation system 190 is coupled to (e.g., integrally formed with, attached to, etc.) the intake canister 170. The roll-off actuation system 190 includes a housing, shown as actuator housing 192, and a pull-string, shown as pull-string 194. According to an exemplary embodiment, the actuator housing 192 includes a rotational member that facilitates unwinding (e.g., in response to a wearer pulling on the pull-string 194, etc.) and winding the pull-string 194 (e.g., in response to the wearer releasing the pulled pull-string 194, etc.). According to an exemplary embodiment, the roll-off actuation system 190 is configured to dispense a new portion of the film strip 198 (e.g., a clean portion, an unused portion, etc.) from the roll of film stored in the supply canister 180 across the lens 80 in response to a wearer of the goggles 10 pulling on the pull-string 194. The intake canister 170 is configured to receive and store (e.g., roll up, etc.) the old portion of the film strip 198 (e.g., the used portion, the dirty portion, etc.).

In some embodiments, the goggles 10 additionally or alternatively include a rip-off system. For example, a tab may be positioned on each of the right flange 90 and the left flange 94 of the lens 80 and/or on each of the right outrigger 110 and left outrigger 120. The tabs may be configured to facilitate coupling one or more rip-off film sheets across the lens 80 and the quick removal thereof (e.g., when the rip-off film sheet becomes dirty obscuring the line of sight of the wearer, etc.).

In some embodiments, the googles 10 additionally or alternatively include an attachment system for a light (e.g., a flashlight, an led light, etc.). The attachment system for the light may be positioned on the right outrigger 110, the left outrigger 120, the frame 20, and/or still otherwise positioned. The attachment system for the light may be configured to receive and hold a light-emitting device (e.g., a flashlight, LEDs, etc.) on the goggles 10 to illuminate a visible area in front of and/or to the sides of the wearer.

In some embodiments, the googles 10 additionally or alternatively include an attachment system for a camera (e.g., an action camera, a still-photography camera, a GoPro®, etc.). The attachment system for the camera may be positioned on the right outrigger 110, the left outrigger 120, the frame 20, and/or still otherwise positioned. In one embodiment, the attachment system for the camera is positioned on one side of the goggles 10 (e.g., on the right outrigger 110, on the right frame end 60, on the left outrigger 120, on the left frame end 70, etc.) to receive and hold a camera device for capturing photos and/or video of the wearer performing some action (e.g., riding a dirt bike, etc.). In other embodiments, the attachment system for the camera is positioned on both sides of the goggles 10 (e.g., on the right outrigger 110 and the left outrigger 120, on the right frame end 60 and the left frame end 70, etc.) to receive and hold two camera devices to the googles 10. According to an exemplary embodiment, the goggles 10 having two camera devices attached thereto facilitates three-dimensional and/or virtual filming of the wearer's actions.

In some embodiments, the googles 10 additionally or alternatively include an attachment system for an integrated heads up display (HUD). The attachment system for the HUD may be positioned on the right outrigger 110, the left outrigger 120, the frame 20, and/or still otherwise positioned. The attachment system for the HUD may be configured to receive and hold a HUD on the goggles 10 to provide various information across the lens 80 for viewing by the wearer of the goggles 10 (e.g., directions, notifications, vehicle information, etc.).

It is important to note that the construction and arrangement of the elements of the systems, methods, and/or apparatuses as shown in the exemplary embodiments are illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements. It should be noted that the elements and/or assemblies of the enclosure may be constructed from any of a wide variety of materials that provide sufficient strength or durability, in any of a wide variety of colors, textures, and combinations.

Embodiments have been described in connection with the accompanying drawings. However, it should be understood that the figures are not drawn to scale. Distances, angles, shapes, etc. are merely illustrative and do not necessarily bear an exact relationship to actual dimensions and layout of the articles that are illustrated. In addition, the foregoing embodiments have been described at a level of detail to allow one of ordinary skill in the art to make and use the articles, parts, different materials, etc. described herein. A wide variety of variation is possible. Articles, materials, elements, and/or steps can be altered, added, removed, or rearranged. While certain embodiments have been explicitly described, other embodiments will become apparent to those of ordinary skill in the art based on this disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or configurations are in any way required for one or more embodiments. The terms “comprising,” “including,” “having,” and the like are synonymous and are used inclusively, in an open-ended fashion, and do not exclude additional elements, features, acts, operations, and so forth. The term “consisting essentially of” can be used anywhere where the terms comprising, including, containing or having are used herein, but consistent essentially of is intended to mean that the claim scope covers or is limited to the specified materials or steps recited and those that do not materially affect the basic and novel characteristic(s) of the claimed invention. Also, the term “consisting of” can be used anywhere where the terms comprising, including, containing or having are used herein, but consistent of excludes any element, step, or ingredient not specified in a given claim where it is used.

Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

Additionally, in the subject description, the word “exemplary” is used to mean serving as an example, instance, or illustration. Any embodiment or design described herein as “exemplary” is not necessarily to be construed as preferred or advantageous over other embodiments or designs. Rather, use of the word exemplary is intended to present concepts in a concrete manner. Accordingly, all such modifications are intended to be included within the scope of the present inventions. Other substitutions, modifications, changes, and omissions may be made in the design, operating conditions, and arrangement of the preferred and other exemplary embodiments without departing from scope of the present disclosure or from the spirit of the appended claims.

What is claimed is:

1. A goggle, comprising:
a frame having a first lateral portion, an opposing second lateral portion, an upper portion, and a lower portion defining a cavity therebetween;
a lens disposed across the cavity; and
a locking system configured to selectively secure the lens to the frame, the locking system including:
a first locking member releasably coupled to the first lateral portion of the frame; and
a second locking member releasably coupled to the opposing second lateral portion of the frame.

2. The goggle of claim 1, wherein the first lateral portion of the frame defines a first face having a first coupler extending therefrom, and the opposing second lateral portion of the frame defines a second face having a second coupler extending therefrom.

3. The goggle of claim 2, wherein the lens includes a first flange shaped to correspond with the first face of the first lateral portion, the first flange defining a first aperture positioned to correspond with the first coupler such that the first coupler extends therethrough.

4. The goggle of claim 3, wherein the first locking member includes a first engagement portion configured to interface with the first coupler such that the first coupler extends through the first aperture of the lens and the first engagement portion of the first locking member to selectively secure the first locking member to the first lateral portion of the frame and the first flange of the lens between the first face of the first lateral portion of the frame and the first engagement portion of the first locking member.

5. The goggle of claim 4, wherein the lens includes an opposing second flange shaped to correspond with the second face of the opposing second lateral portion, the opposing second flange defining a second aperture positioned to correspond with the second coupler such that the second coupler extends therethrough.

6. The goggle of claim 5, wherein the second locking member includes a second engagement portion configured to interface with the second coupler such that the second coupler extends through the second aperture of the lens and the second engagement portion of the second locking member to selectively secure the second locking member to the opposing second lateral portion of the frame and the opposing second flange of the lens between the second face of the opposing second lateral portion of the frame and the second engagement portion of the second locking member.

7. The goggle of claim 1, wherein the upper portion of the frame defines (i) a first engagement surface and a first recess positioned towards the first lateral portion of the frame and (ii) a second engagement surface and a second recess positioned towards the opposing second lateral portion of the frame.

8. The goggle of claim 7, wherein the first locking member includes a first arm shaped to correspond with the first engagement surface, and wherein the second locking member includes a second arm shaped to correspond with the second engagement surface.

9. The goggle of claim 8, wherein the first arm has a first locking portion positioned along the first arm including a first latch that interfaces with the first recess, and wherein the second arm has a second locking portion positioned along the second arm including a second latch that interfaces with the second recess.

10. The goggle of claim 9, wherein the first locking portion includes a first extension and the second locking portion includes a second extension, the first extension and the second extension positioned to extend along a front surface of the lens holding a top edge of the lens against the upper portion of the frame.

11. The goggle of claim 1, wherein the first locking member defines a first depression and the second locking member defines a second depression.

12. The goggle of claim 11, further comprising at strap having (i) a first end including a first connector configured to releasably secure the first end of the strap to the first locking member when the first connector is received within the first depression and (ii) a second end including a second connector configured to releasably secure the second end of the strap to the second locking member when the second connector is received within the second depression.

13. The goggle of claim 1, further comprising a roll-off system including:
a first canister configured to store a roll of film and facilitate dispensing a strip of film from the roll of film; and
a second canister configured to intake the strip of film;
wherein the roll-off system is configured to dispense the strip of film from the roll of film across the lens from the first canister to the second canister.

14. The goggle of claim 13, wherein the first canister is attached to the first locking member and the second canister is attached to the second locking member.

15. The goggle of claim 13, wherein the first canister and the second canister are attached to opposing ends of the lens.

16. An attachment system configured to selectively secure a lens to a frame of an eyewear, the attachment system comprising:
a first attachment member configured to releasably couple to a first lateral portion of the frame, the first attachment member including a first engagement portion configured to interface with a first coupler extending from the first lateral portion of the frame to selectively secure a first end of the lens between the first lateral portion and the first engagement portion; and
a second attachment member configured to releasably couple to an opposing second lateral portion of the frame, the second attachment member including a second engagement portion configured to interface with a second coupler extending from the opposing second lateral portion of the frame to selectively secure an opposing second end of the lens between the opposing second lateral portion and the second engagement portion.

17. The attachment system of claim 16, wherein the first attachment member includes a first arm extending from the first engagement portion, the first arm shaped to correspond with a first surface of an upper portion of the frame, and wherein the second attachment member includes a second arm extending from the second engagement portion, the second arm shaped to correspond with a second surface of the upper portion of the frame.

18. The attachment system of claim 17, wherein the first arm includes a first locking portion, the first locking portion including at least one of (i) a first frame engagement portion configured to correspond with a first recess defined by the upper portion of the frame and (ii) a first lens engagement portion configured to extend downward and along a front surface of the lens, and wherein the second arm includes a second locking portion, the second locking portion including at least one of (i) a second frame engagement portion configured to correspond with a second recess defined by the upper portion of the frame and (ii) a second lens engagement portion configured to extend downward and along the front surface of the lens.

19. The attachment system of claim 17, wherein the first engagement portion and the first arm are integrally formed such that the first attachment member has a unitary structure, and wherein the second engagement portion and the second arm are integrally formed such that the second attachment member has a unitary structure.

20. An eyewear, comprising:
a frame having a first lateral portion including a first coupler and an opposing second lateral portion including a second coupler;
a lens having a first end defining a first aperture and an opposing second end defining a second aperture, the first aperture and the second aperture positioned to receive the first coupler and the second coupler, respectively; and
an attachment system configured to selectively secure the lens to the frame, the attachment system including:
a first attachment member including a first engagement portion configured to interface with the first coupler such that the first coupler extends through the first aperture of the lens and the first engagement portion of the first attachment member to selectively couple the first attachment member to the first lateral portion of the frame and the first end of the lens between the first lateral portion of the frame and the first engagement portion of the first attachment member; and
a second attachment member including a second engagement portion configured to interface with the second coupler such that the second coupler extends through the second aperture of the lens and the second engagement portion of the second attachment member to selectively couple the second attachment member to the opposing second lateral portion of the frame and the opposing second end of the lens between the opposing second lateral portion of the frame and the second engagement portion of the second attachment member.
